(19) United States
(12) Patent Application Publication
(10) Pub. No.: US 2024/0269354 A1
(43) Pub. Date: Aug. 15, 2024

(54) CARDIOTHORACIC DRAINAGE DEVICE

(71) Applicant: REDAX S.P.A., Poggio Rusco (IT)

(72) Inventor: Lucio GIBERTONI, Mirandola (IT)

(21) Appl. No.: 18/569,509

(22) PCT Filed: Dec. 21, 2022
(86) PCT No.: PCT/EP2022/087297

§ 371 Date: Dec. 12, 2023

(30) Foreign Application Priority Data
May 5, 2022 (IT) 102022000009173

(51) Int. Cl. A61M 1/04; A61M 1/00; A61M 27/00

(52) U.S. Cl. CPC A61M 1/04; A61M 1/64; A61M 1/742; A61M 27/00; A61M 2205/3375; A61M 2205/3389; A61M 2205/3592; A61M 2205/502

(57) ABSTRACT

A cardiothoracic drainage device
includes a collection unit adapted to contain a cardiothoracic liquid drained from the chest of a patient;
a main unit adapted to contain the components of the drainage device and to measure the cardiothoracic liquid contained in the collection unit; and
a connection tube associated with the collection unit, with the main unit and with the drainage tube.

The main unit includes inside it:
a suction pump;
a normally closed electric valve;
an interface component adapted to receive inputs by the user and to supply outputs for the user;
an electronic control and management component adapted to control the functions of the drainage device; and
a pressure sensor component operatively connected to the electronic control and management component.

### CROSS-REFERENCE TO RELATED APPLICATIONS

This application is a 35 U.S.C. § 371 National Stage patent application of PCT/EP2022/087297, filed on 21 Dec. 2022, which claims the benefit of Italian patent application no. 102022000009173, filed on 5 May 2022, the disclosures of which are incorporated herein by reference in their entirety.

### TECHNICAL FIELD

The present disclosure relates to a cardiothoracic drainage device.

### BACKGROUND

In the field of cardiothoracic drainage, following surgery and/or traumas, it is necessary to apply drainage systems that allow the evacuation of excess fluids accumulated in body cavities, which can give rise to various kinds of complications.

In the course of the past century, drainage systems have evolved, starting from the simplest embodiments using reusable glass flasks to modern monolithic disposable systems in which the various functions are integrated in a single device.

Currently, the state of the art in cardiothoracic drainage has seen the recent introduction of digital devices for support and/or integrated with the respective containers, with the goal of being able to measure and represent objectively the quantities that the physician monitors over time to assess the progress of the postoperative course.

In greater detail, in the postoperative drainage of the cardiothoracic patient there are three basic parameters whose value determines the end of postoperative treatment and the consequent possibility to remove the drainage from a patient: air leakage from the pleural cavity, intrapleural pressure, and collection of liquids drained over time.

Air leakage is the most important parameter in the case of lung surgery and/or in all those cases where pleural involvement has occurred.

Indeed, suturing the lung, despite advanced surgical techniques and the instrumentation available, remains a process that easily leaves a residual postoperative leakage that can last from a few hours to several weeks, depending on the type of surgery performed and on the clinical conditions of the patient.

In traditional systems, air leaks are monitored visually and qualitatively by observing the presence of bubbles in a specially adapted water seal valve.

Recently, the appearance of digital drainage systems has enabled measurement of this parameter by using flowmeters or with a volumetric calculation method (performing conversion between the activation time of a suction pump and the corresponding aspirated volume of air).

As regards intrapleural pressure, it is localized between the two membranes (parietal and visceral) and is subject to variations that may indicate the patient's clinical status.

In fact, intrapleural pressure is closely tied to the state of expansion of the lung within the rib cage: in physiologically normal conditions, this pressure assumes an average negative value around −6/−8 cm H2O, with oscillations that are synchronous with the patient's breathing, whereas in a compromised lung (e.g., following surgery) this value may be subject to oscillations of high amplitude.

In the presence of conditions of increased resistance of the pneumatic connections (typically the case of the tube for connection to the catheter that is full of drained liquid), intrapleural pressure may assume highly positive values, generated by the effort made by the patient himself to evacuate air from the pleural cavity.

Finally, the measurement of liquid collection takes on different connotations depending on whether lung surgery rather than cardiac surgery is considered.

More specifically, in the former case, the figure related to the liquid drained over the last 24 hours becomes more important because it is the parameter that contributes to the physician's decision to remove the catheter.

Differently, in the second case, the drained liquid is monitored closely and frequently in the first twenty-four hours, since any postoperative bleeding must be detected immediately; monitoring is considered less crucial in the following days.

All the known types of cardiothoracic drainage device described up to this point are affected by the problem mentioned earlier regarding the measurement of intrapleural pressure.

For example, with particular reference to bottle-type cardiothoracic drainage devices, the patient tube tends very frequently to form bends in which the drainage liquid accumulates.

In greater detail, the height in centimeters of the column of liquid “h” that is formed corresponds to the numerical value, expressed in cm of H2O, of the positive pressure that the patient must exert in order to overcome hydraulic resistance and expel air from the chest.

For traditional devices, the value “h” can reach and exceed 30 cm of H2O, with the consequent risk of formation of a tension pneumothorax, often concomitant with subcutaneous emphysema that must necessarily be treated.

Periodic emptying of the connection tube by nurses occurs occasionally and therefore it is not sufficient to reduce the consequences resulting from the presence of the column of liquid.

With the entry into the market of so-called “digital” cardiothoracic drainage devices, an example of which is described in U.S. Pat. No. 8,486,051, dual lumen tubes have made their appearance in which the first lumen, generally of a larger diameter, is assigned to the drainage of fluids (air and liquids) and the second lumen is used to monitor the pressure at the connection with the catheter, which is compared with the pressure present in the drainage container.

If the former is lower than the latter, this means that liquid is present in the tube; at this point suction can be increased until the main lumen is emptied.

One drawback of these known types of device, related to the process just described, is that when the tube becomes empty the increase in suction cannot be reduced quickly enough and therefore it is transmitted directly and abruptly to the patient and can reach values of more than −70 cm H2O, with the possibility of causing damage to the lung parenchyma.

Another drawback, which affects devices of the known type, concerns the measurement of the drained liquids.

In greater detail, when using traditional drainage devices, personnel are forced to make a note of the level reached each time by the liquid in the container and calculate the difference with respect to the previous measurement in order to understand its progress over time.

In these devices, the measurement is made by means of a graduated scale directly on the collection vessel.

Inevitably, the resolution of the graduated scales conditions the measurement made.

Typically, this resolution is often achieved by subdividing the receptacle into multiple small sectors that communicate with each other by overflow from the first one to the following ones.

Health care personnel, in order to obtain information on the progress of liquid collection over time, must necessarily make manual readings and annotations at various times of the day, and in the event of accidental tipping of the collection vessel, the collected liquid becomes distributed randomly in the various graduated sectors, forcing the personnel to perform further calculations to obtain the necessary information.

In so-called “digital” cardiothoracic drainage devices, the detection described above is carried out primarily with sensors with capacitive coupling, an example of which is described in U.S. Pat. No. 9,333,281, in which the measurement occurs on a vertical side of the collection vessel.

This process allows to have a digital reading of the level of the liquid in the container in real time and consequently the calculation of the level variation over a given time.

However, even this type of cardiothoracic drainage device of the known type is not without drawbacks, which include the fact that the measurement with sensors with capacitive coupling comprises the need to make the measurement on a vertical side of the drainage system, resulting in a strong dependence of the measurement on any inclination of the drainage with respect to the horizontal plane.

Moreover, measuring on the vertical side necessarily increases the width of the system as a whole as the collection capacity of the container increases.

Finally, the presence of the sensors on the vertical side of the digital device makes it necessary for this wall to be rectilinear and such as to allow firm coupling to the container.

Therefore, such container will have a design imposed by the particular geometry.

Finally, among known types of cardiothoracic drainage device there are devices that represent the data related to the patient's postoperative course on a display located directly on the device itself.

Currently, the size of such displays (color LCD or TFT) varies from 3 to 4.5 inches, but their width is limited by the structure of the drainage device and by the need to keep low the overall space occupation and weight.

As a result, reading the data is difficult, especially in view of the fact that the degree of sophistication of the devices is increasing continually and so is the amount of information shown.

In order to access all the data provided and the functions of digital drainages it is often necessary to resort to multi-step procedures, using drop-down menus, which entail difficulty in management by medical and nursing staff.

### SUMMARY

The aim of the present disclosure is to provide a cardiothoracic drainage device that is capable of overcoming the drawbacks and limitations noted above.

Within this aim, the present disclosure provides a cardiothoracic drainage device that is capable of solving the drawbacks related to the measurement of the drained liquids which are associated with currently used technologies.
the present disclosure also provides a cardiothoracic drainage device that can have high immunity to container tilting, facilitating its transport and reducing its overall space occupation.
the present disclosure further provides a cardiothoracic drainage device that is capable of solving the problem of the accumulation of liquids in the tube for connection to the patient, with a consequent increase in the pressure required for the patient to evacuate air from the pleural cavity.
the present disclosure also provides a cardiothoracic drainage device that is capable of solving the drawbacks related to the poor visibility of the data shown and to the difficulty of managing the user interface that are present in the background art.

This aim, as well as the advantages mentioned and others that will become better apparent hereinafter, are achieved by providing a cardiothoracic drainage device comprising:
a collection unit adapted to contain a cardiothoracic liquid drained from the chest of a patient;
a main unit adapted to contain the components of said drainage device and to measure the cardiothoracic liquid contained in said collection unit, said collection unit being associable with said main unit; and
a connection tube which can be associated with said collection unit, with said main unit and with the drainage tube partially inserted in the thoracic cavity of said patient from which said cardiothoracic liquid is to be drained;

characterized in that said main unit comprises inside it:
a suction pump, which can be operatively associated with said connection tube by means of a pneumatic circuit in order to create the drainage suction pressure and keep it adjusted;
a normally closed electric valve, which is inserted in said pneumatic circuit and adapted to ensure the seal of said pneumatic circuit when said suction pump is not moving;
interface means adapted to receive inputs by the user and to supply outputs for the user;
electronic control and management means adapted to control the functions of said drainage device; and
pressure sensor means operatively connected to said electronic control and management means for the monitoring of said drainage suction pressure and of the intrapleural pressure of said patient.

### BRIEF DESCRIPTION OF THE DRAWINGS

Further characteristics and advantages of the disclosure will become better apparent from the description of a preferred but not exclusive embodiment of a cardiothoracic drainage device, illustrated by way of non-limiting example with the aid of the accompanying drawings, wherein:

FIG. 1 is a front perspective view of the drainage device according to the present disclosure;

FIG. 2 is a front perspective view of the main unit of the drainage device shown in FIG. 1;

FIG. 3 is a transverse sectional view of the main unit shown in FIG. 2;

FIGS. 4 to 8 are five transverse sectional views showing sequentially the steps of association of the liquid collection unit of the drainage device according to the disclosure with the main unit shown in the preceding figures;

FIG. 9 is a perspective view of the connection tube of the drainage device shown in the preceding figures;

FIG. 10 is a perspective view of the drainage device according to the disclosure together with an external display device; and

FIGS. 11 to 14 are four views of the information that can be shown on the display of the main unit shown in the preceding figures.

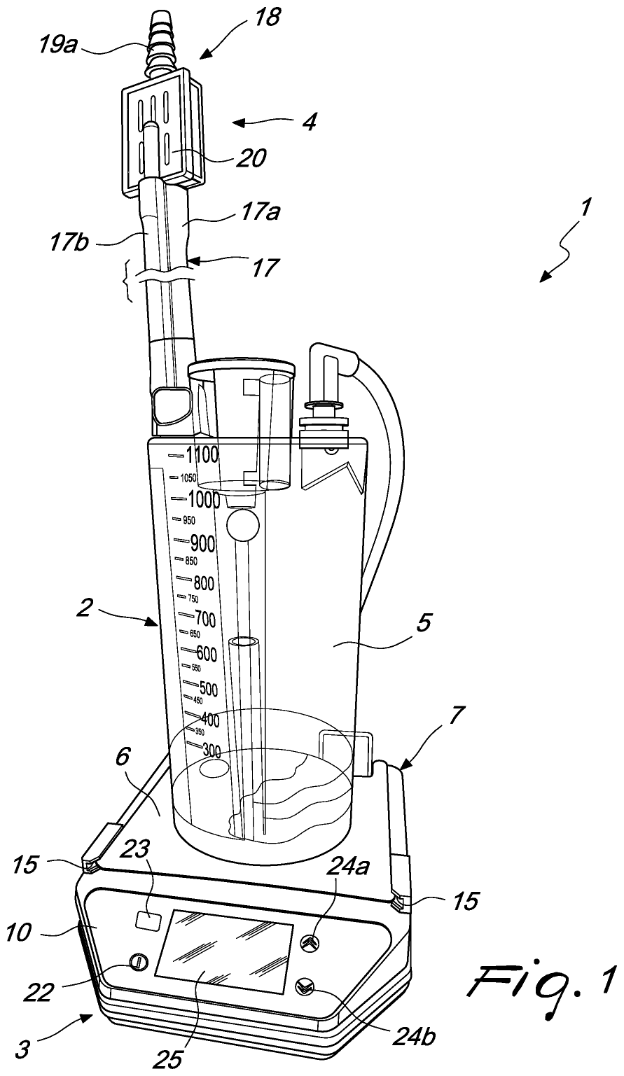

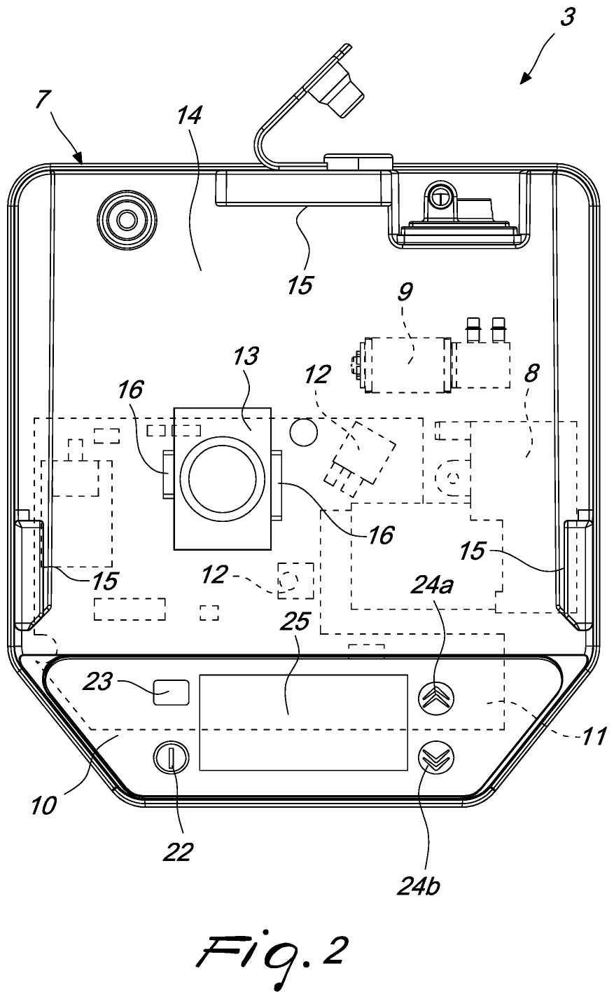

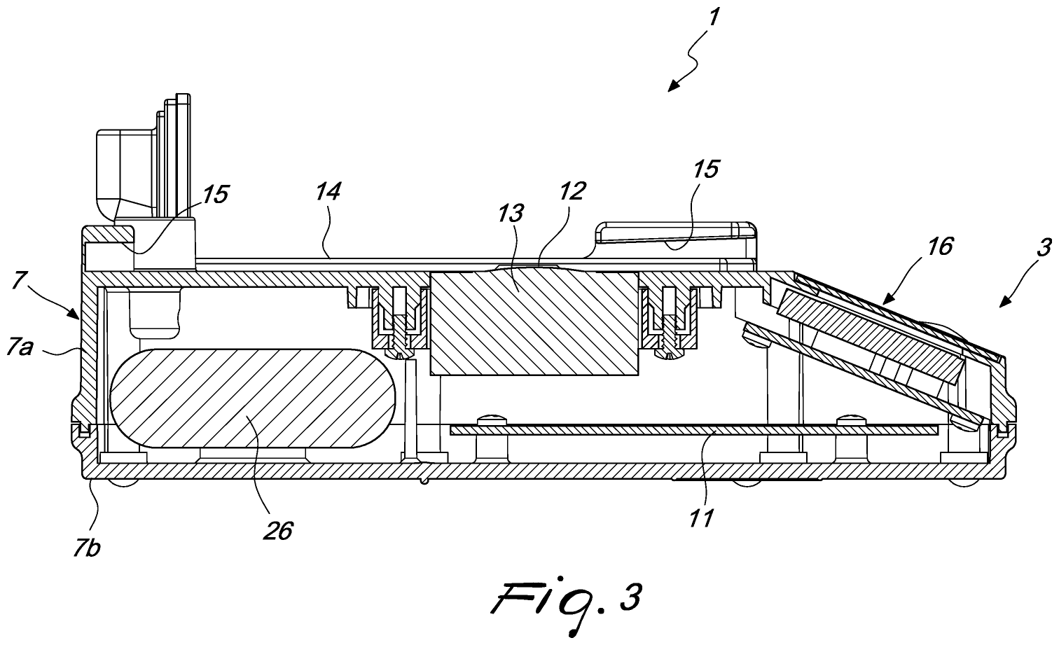

### DETAILED DESCRIPTION OF THE DRAWINGS

With reference to the figures, the cardiothoracic drainage device, generally designated by the reference numeral 1, comprises: a collection unit 2, a main unit 3, and a connection tube 4.

In greater detail, the collection unit 2 substantially comprises a drainage container 5, for example provided with a graduated scale on its lateral surface, adapted to contain cardiothoracic liquid drained from a patient's chest, and is provided with a series of appendages 6 which, as will become better apparent hereinafter, are intended to firmly and securely associate the collection unit 2 with the main unit 3.

Conveniently, the main unit 3, which is adapted to contain the components of the drainage device 1 and to measure the cardiothoracic liquid contained in the collection unit 2, comprises a hermetic external enclosure 7, made of impact-resistant plastic material, composed of two half-shells—of which one is an upper one 7a and one is a lower one 7b—which are mutually associated by virtue of screw means and between which a gasket is interposed so as to avoid the penetration of liquids and dust from the outside.

According to the disclosure, the main unit 3 comprises within it:
a suction pump 8, which can be operatively associated with the connection tube 4 by means of a pneumatic circuit in order to create the drainage suction pressure and keep it adjusted;
a normally closed electric valve 9, which is inserted in the pneumatic circuit and is adapted to ensure the seal thereof when the suction pump 8 is not moving;
interface means 10 adapted to receive inputs by the user and to supply outputs for the user;
electronic control and management means 11, constituted by an electronic board provided with a microprocessor, which are adapted to control the functions of the drainage device 1; and
pressure sensor means 12 operatively connected to the electronic control and management means 11 for the monitoring of the drainage suction pressure and of the intrapleural pressure of the patient.

More specifically, the suction pump 8 is preferably of the diaphragm type the rotation rate of which can be adjusted by means of a variation in the power supply voltage.

Advantageously, the main unit 3 further comprises at least one ultrasonic sensor 13 placed in its portion designed to engage the bottom of the collection unit 2 to measure the amount of cardiothoracic liquid collected in the collection unit 2 itself, i.e., in the drainage container 5.

In greater detail, the external enclosure 7 forms on its back a receptacle 14 for accommodating the collection unit 2 which is provided with interlocking profiles 15 suitable to engage corresponding appendages 6, mentioned earlier, which are formed on the bottom of the collection unit 2 in such a way as to form a stable and integral coupling between the main unit 3 and the collection unit 2.

Conveniently, the ultrasonic sensor 13 is located in said accommodation receptacle 14.

In greater detail, due to the nature of the collected fluids, which are often contaminated and therefore entail a high biohazard, and due to the consequent need to use disposable containers, the ultrasonic sensor 13 could not be placed inside the drainage container 5.

This would in fact complicate the disposal procedures—electronic waste would be attached to biologically hazardous waste—and would increase the cost of the disposable device.

Advantageously, the main unit 3 comprises two ribs 16 which are located laterally to the ultrasonic sensor 13 and protrude in the direction of the collection unit 2 with respect to the resting plane formed by the accommodation receptacle 14 so as to avoid damage to the ultrasonic sensor 13.

In fact, the top of the ultrasonic sensor 13 is made of a soft material, usually silicone, which as a result of repeated use of the drainage device 1, with multiple couplings and uncouplings between the collection unit 2 and the main unit 3, could wear out.

Moreover, in this manner optimal contact between the ultrasonic sensor 13 and the collection unit 2, with consequent effective transmission of sound waves from the former toward the latter, is ensured.

As regards the connection tube 4, it can be associated with both the collection unit 2 and the main unit 3 and can be associated with the drainage tube partially inserted into the thoracic cavity of the patient from which the cardiothoracic liquid is to be drained.

In greater detail, the connection tube 4 comprises:
a dual lumen tube 17, made of a flexible plastic material, which has a main lumen 17a designed to convey the liquids and a secondary lumen 17b which can have a diameter that is equal to or smaller than that of the main lumen 17a and is dedicated exclusively to the evacuation of the air that arrives from the chest of the patient;
a connector 18 associated with a first end of the dual lumen tube 17 and provided with a first tapered connector 19a for connection to the drainage tube and with a chamber 20 in which a hydrophobic filtering membrane 21 is present so as to allow the separation of the air from the liquid; and
a second connector 19b, associated with a second end of the dual lumen tube 17 which is opposite the first end, and of the quick release (lock-in) type so as to allow the connection of the connection tube 4 to the collection unit 2 so as to allow quick and safe replacement of the latter without acting proximate to the drainage tube.

In greater detail, the connection tube 4 thus constituted primarily allows to evacuate the air of the patient through the dedicated secondary lumen 17b, which is independent and always pervious by virtue of the presence of the hydrophobic filter membrane 21.

In fact, the patient's fluids, which arrive from the chest, are typically composed of air and liquids and when they enter the chamber 20 separation is performed: the liquid is conveyed into the main lumen 17a while the air passes through the hydrophobic filtering membrane 21, since it is a path with less resistance and arrives directly in the container through the secondary lumen 17b.

The presence of this pathway, which is always pervious, in addition to the evacuation of the air from the patient's chest allows intrapleural pressure to be measured directly and precisely, without interference due to the liquid.

In other words, the secondary lumen 17b and the always-pervious pathway, which it forms, provide a twofold result: the first comprises evacuating the patient's air safely and with low resistance, and the second comprises being able to make the intrapleural pressure measurement easily in the collection container, since it corresponds exactly to the pressure present at the connector 18, i.e., at the end of the drainage tube.

In fact, without this privileged pathway, intrapleural pressure would have to be measured directly on the connector 18, for example by providing a specific fitting, thus with more inconvenience and complications.

As regards the interface means 10, they comprise a control keypad, constituted by a power button 22, a functional page rotation button 23 and two buttons 24a and 24b for drainage suction pressure adjustment, and a display 25 of the monochrome or color OLED type or equivalent, which allows to have optimal readability at all times under any external light conditions.

Finally, to complete the drainage device 1, the main unit 3 may comprise electric power storage means 26, i.e., one or more rechargeable batteries, for the operation of the drainage device 1 while disconnected from the electrical grid.

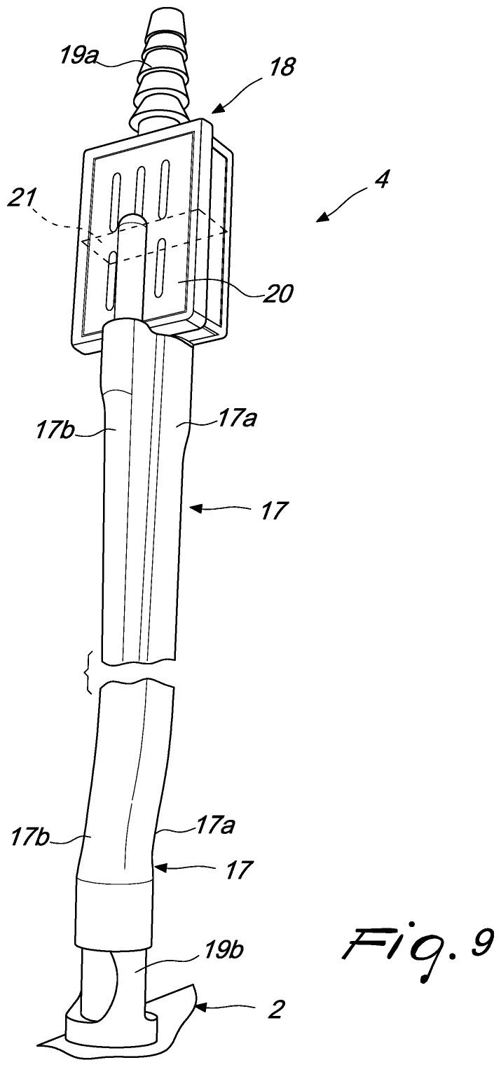

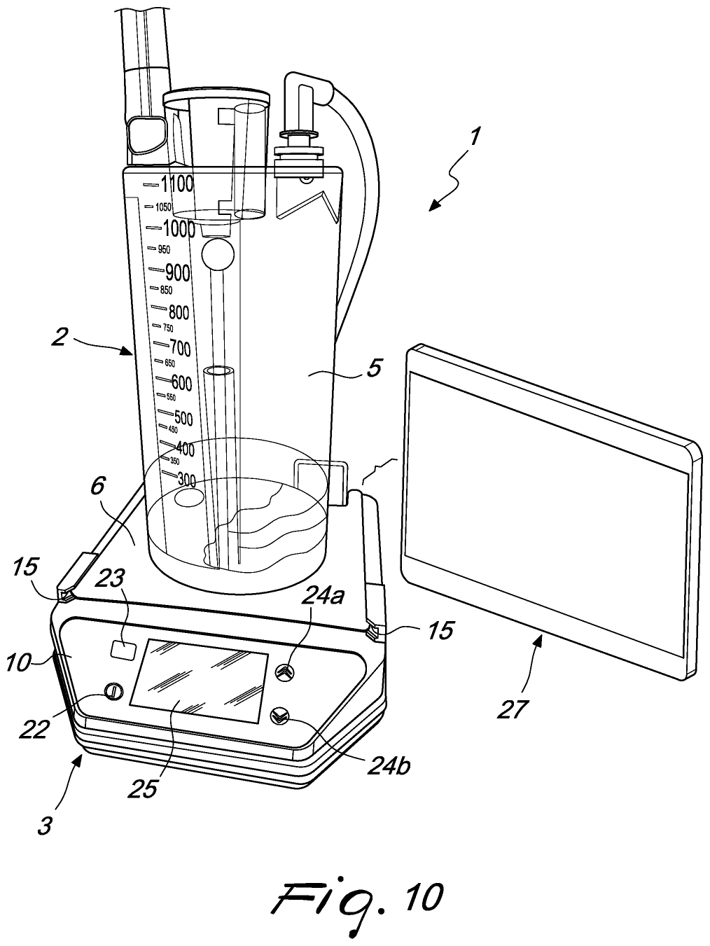

All the data measured by the drainage device 1 are stored and can be transferred to an external device 27 via a wireless telecommunications module, preferably of the Bluetooth® or Wi-Fi type, as shown in FIG. 10.

This internal circuit expands the physician's possibilities to manage the patient's clinical data.

In fact, the progress of the postoperative course can be displayed on the external device 27, such as a PC or tablet, greatly increasing the readability of the information.

Moreover, the data history can be entered in the patient's digital medical record and quickly correlated with all the other clinical elements, such as blood tests, radiographic investigation results, etc.

Therefore, the physician is able to have a complete picture of the patient's clinical status and consequently make a more accurate diagnosis.

Finally, the data of a patient can be sent remotely to another physician or team for expanded consultation.

The operation of the drainage device 1 according to the present disclosure is clear and evident from what has been described so far.

In particular, it should be noted that the microprocessor with which the electronic control and management means 11 are equipped has various inputs, including: the sensor means 12 for measuring intrapleural pressure, the ultrasonic sensor 13 for measuring the level of the liquid, and the control keyboard from which it accepts user commands.

The microprocessor outputs to the display 25 the information related to operation and to the measurements taken.

Moreover, an additional sensor monitors the pressure and provides its value to the microprocessor, which accordingly commands the activation of the suction pump 8 in order to maintain the user's desired negative value.

Finally, an encoder provides the microprocessor with the number of revolutions made by the suction pump 8, by means of which the volume of air lost from the patient's chest is calculated.

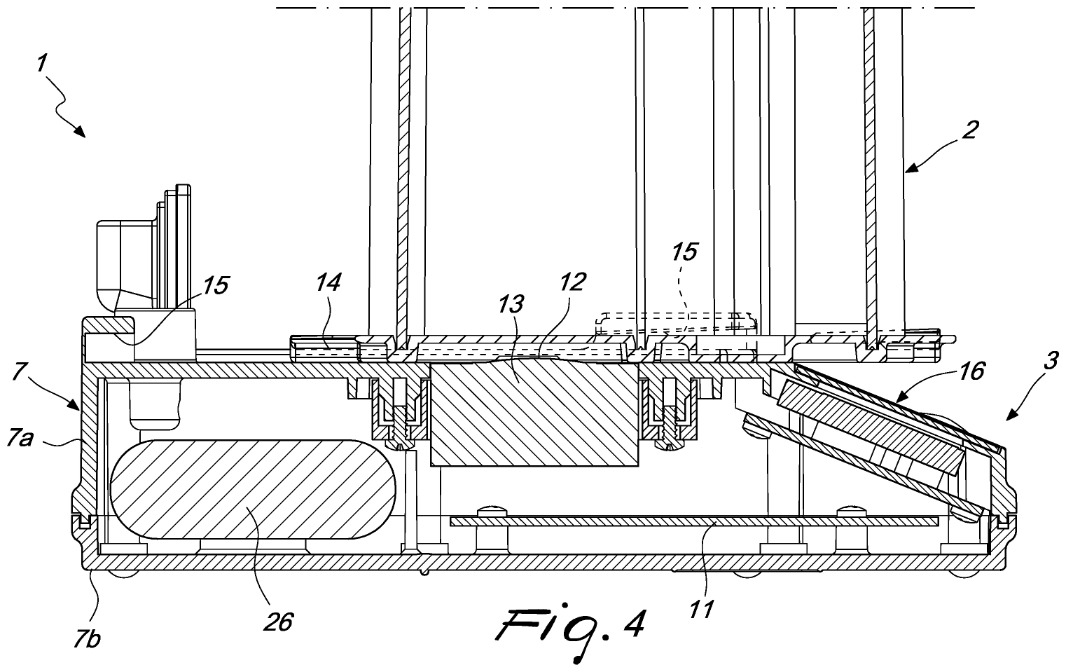

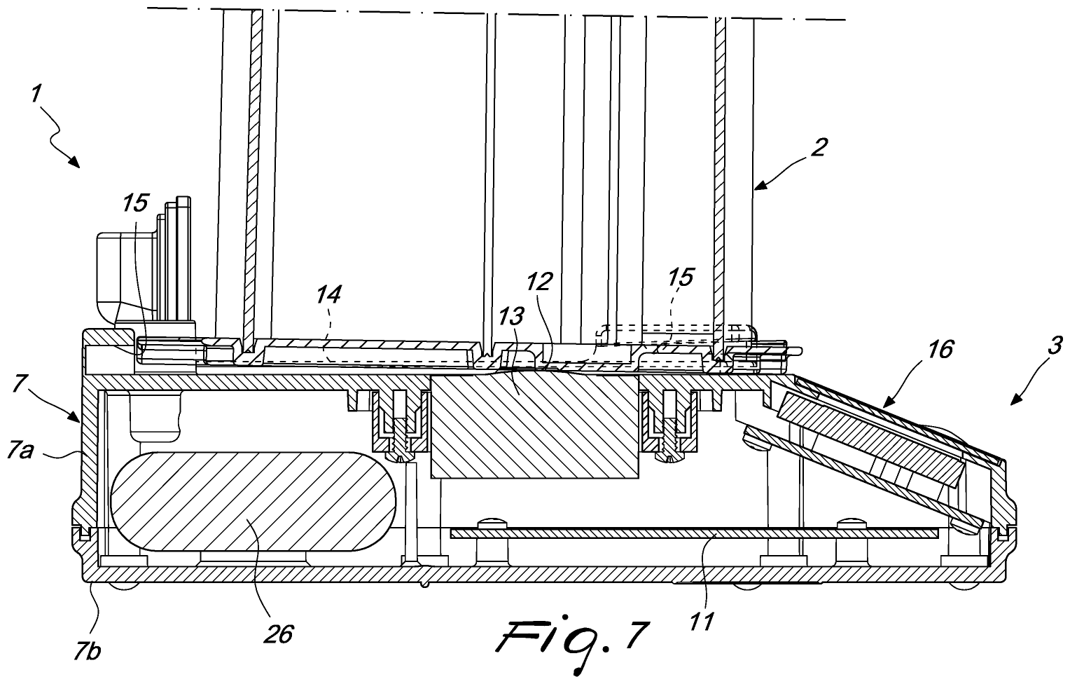

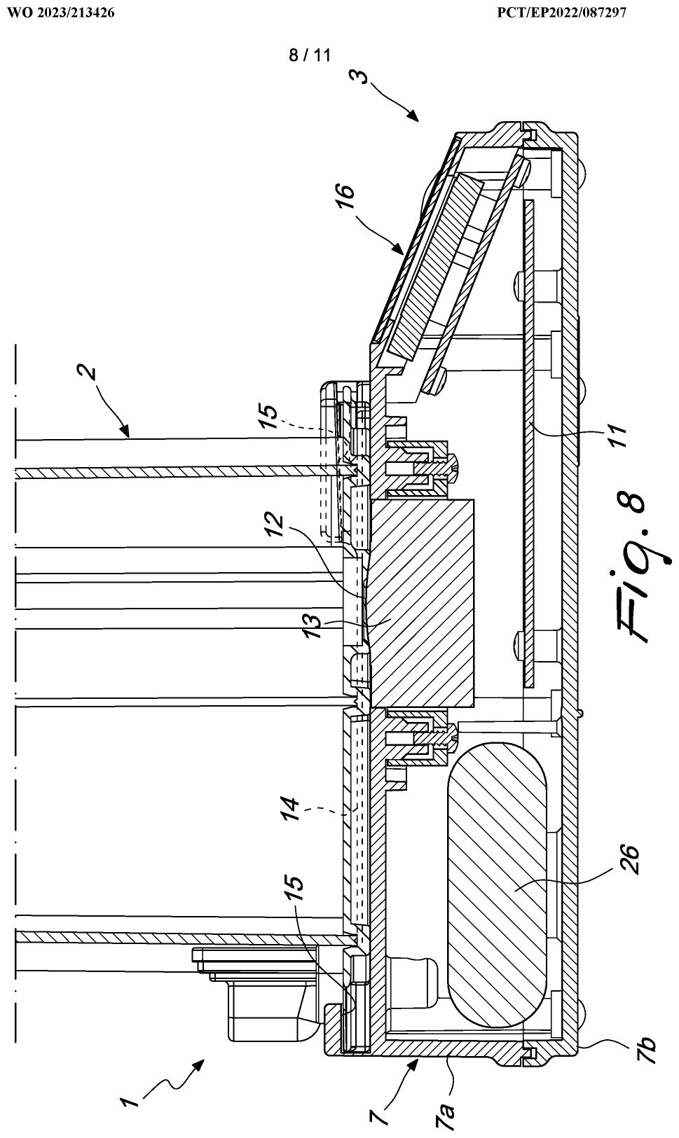

With particular reference to FIGS. 4 to 8, in which the steps of mutual association between the main unit 3 and the collection unit 2 are shown sequentially, the user places the drainage container 5 on the upper surface of the main unit 3 at the accommodation receptacle 14, starting from the front side.

During this step, the base of drainage container 5 rests completely on the back of the main unit 3, as shown in FIG. 4.

The user then begins to slide the drainage container 5 toward the back of the main unit 3 while continuing to rest on the back of the main unit, as shown in FIG. 5.

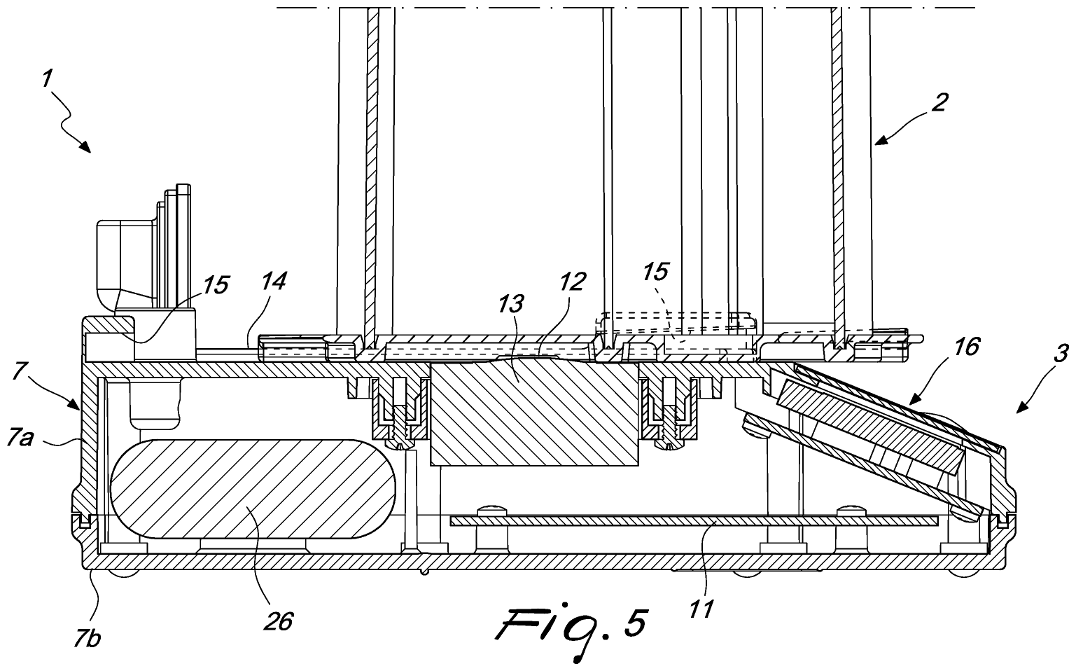

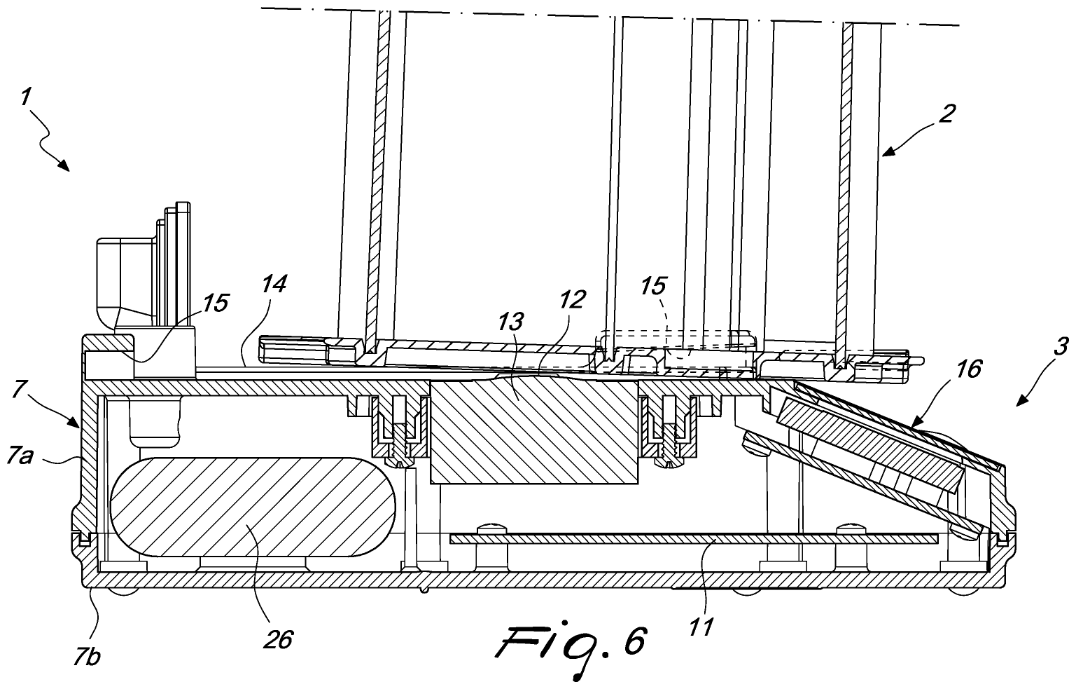

At this point, at the ribs 16 the footing of the drainage container 5, which directly forms the appendages 6, is raised by a small amount, sufficient to avoid the ultrasonic sensor 13 and thus prevent damage to it, as shown in FIG. 6.

Once it has moved beyond the ultrasonic sensor 13, the footing of the drainage container 5 is lowered, coming back into contact with the back of the main unit 3, and while the appendages 6 engage the interlocking profiles 15, the contact section is lowered so that it ends its travel exactly at the ultrasonic sensor 13, as shown in FIG. 7.

Once the collection unit 2 is fully inserted and fixed in the accommodation receptacle 14 of the main unit 3, the specially designed section of the footing of the drainage container 5 is in contact with the ultrasonic sensor 13 and fixing allows to maintain the necessary pressure between the two units so as to have the optimum measurement, as shown in the figure.

It is appropriate to note that, again with particular reference to FIGS. 4 to 8, this mode of insertion furthermore allows to maintain contact between the top of the ultrasonic sensor 3 and the bottom of the collection unit 2 even if the entire drainage device is lifted for transport or to avoid its direct contact with the floor.

For this purpose, the collection unit 2 can be provided with a handle so that it can be carried and with one or two hooks, integrated into the handle itself, to attach it to the patient's bed.

With particular reference to FIGS. 11-14, it should be noted that the drainage unit 1 is designed to measure and display primary information of clinical interest to the physician, in order to be able to assess the patient's postoperative course. The information is shown on the display 25 by means of a series of “pages” that are scrolled in a circular fashion using the functional page rotation button 23 provided for this purpose.

In greater detail, as shown in FIG. 11, there is a HOMEPAGE page that is the main page where functional data, such as selected suction and battery charge status, are primarily displayed.

Moreover, as shown in FIG. 12, there is an AIR LEAK page, which is the page where information about the patient's air leaks is provided.

In particular, the information is presented both as a real-time leakage value and as an average leakage value over the past hour. Both pieces of information are complementary for proper assessment of the patient's postoperative course.

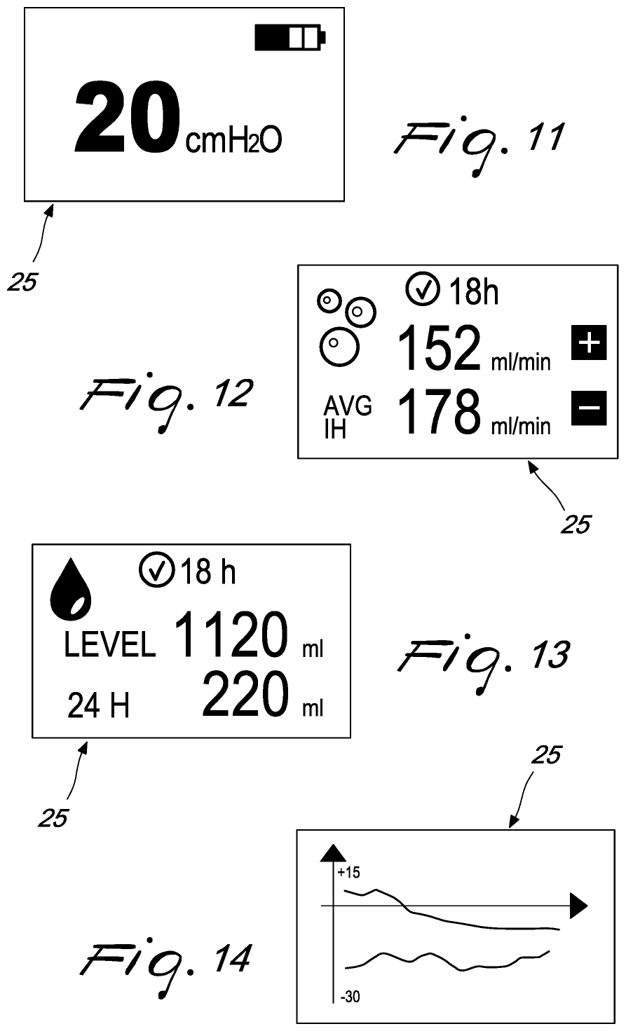

Then, as shown in FIG. 13, a DRAINED LIQUID COLLECTION page is provided.

More specifically, the drainage device 1 is capable of measuring with great precision the level of the liquid in the drainage container 5, showing on the display 25 both its current level and the overall level possibly constituted by the sum of the liquid also collected in the drainage containers 25 used previously and replaced, for example, following their complete filling or as a function of the hospital protocol followed.

Moreover, information regarding the liquid collected in the last twenty-four hours, which is an important parameter for the physician in order to decide on drainage removability, is provided on that page.

Finally, as shown in FIG. 14, an INTRAPLEURAL PRESSURE page is provided.

More specifically, the drainage device 1 measures the pressure that is located between the patient's two pleurae.

This is made possible by the presence of the pressure sensors means 12 connected to the tube 4 for connection to the patient.

The tube, being a dual lumen tube 17 as described above, allows the measurement to be taken directly at the drainage catheter, without any interference, and therefore the value obtained constitutes crucial information for the physician in order to determine the appropriate time for drainage removal.

This data item is provided in the form of a chart, shown indeed in FIG. 14, in which there are two separate lines, representing the trend of maximum and minimum pressures located in the intrapleural space, in relation to the patient's respiratory acts.

Typically in the inspiratory step the pressure tends to be more negative than in the expiratory step, where it can also take on positive values.

The smaller the distance between the two lines, the better the lung expansion and thus the patient's clinical status.

In practice it has been found that the drainage device according to the present disclosure achieves the intended aim and advantages, since it allows to create suction to the patient and keep it adjusted to the value selected by the physician, while measuring the patient's air leakage and converting the running time of the suction pump into the aspirated volume of air.

More specifically, the drainage device according to the present disclosure allows to measure the intrapleural pressure by means of an adapted pressure sensor connected to the drainage catheter via a special dual lumen circuit and to measure the amount of liquid collected in the drainage container by means of an ultrasonic sensor engineered to be in contact with the outside of said container.

In fact, the measurement is made by means of an ultrasonic sensor and without any contact with the drained fluids, either from the bottom (preferred embodiment) or from the top side of the container, reducing the overall dimensions of the resulting drainage device and allowing the freest provision of different shapes and dimensions, since the measurement technology leaves numerous degrees of freedom in design.

More specifically, the greater provided freedom of shapes and space occupations allows the use of containers having differentiated volumes for adult, pediatric, and neonatal patients, for whom it is essential to connect vessels having volumes that are proportionate to the overall volume of the rib cage.

Another advantage of the drainage device according to the present disclosure is that it renders the collection unit integral with the main unit, allowing high immunity to tilting of the drainage container, facilitating its transport and reducing its overall space occupation.

An additional advantage of the drainage device according to the disclosure is that it solves the problem of the accumulation of liquids in the tube for connection to the patient, with consequent increase in the pressure required for the patient to evacuate the air from the pleural cavity.

In fact, the tube for connection to the patient as described allows to never have interference between the liquids and the air, consequently eliminating hydraulic resistance and the risk of pneumothorax.

This configuration allows direct measurement of intrapleural pressure directly at the drainage tube connector and likewise ensures that any suction applied is transferred fully to the patient without any load loss and continuously.

Another advantage of the drainage device according to the present disclosure is that it provides excellent visibility of the data shown, offering a simple and easily understandable user interface.

This advantage is further emphasized by the data transmission card integrated in the device, preferably via Bluetooth or Wi-Fi network, which allows all the information related to the patient's postoperative course to be transferred to an external device (e.g., tablet, smartphone, PC, . . . ).

In this manner, the data are made available and viewable in any format and on a device of the user's choice.

As a result, these data can be easily interpreted by the physician in order to make a diagnosis of the patient's postoperative course.

By means of the external device it is possible to change the settings of the unit and manage the patient's data in multiple ways: e.g., by transferring them to the hospital archive via network connection, sending them to a second physician for consultation, etc.

By means of the connection with the external device, it is no longer necessary to operate directly on the unit, and therefore the user interface can be extremely simplified with respect to currently commercially available devices.

The drainage device thus conceived is susceptible of numerous modifications and variations, all of which are within the scope of the accompanying claims.

All the details may furthermore be replaced with other technically equivalent elements.

In practice, the materials used, so long as they are compatible with the specific use, as well as the contingent shapes and dimensions, may be any according to the requirements and the state of the art.

1-11. (canceled)

12. A cardiothoracic drainage device, comprising:
a collection unit adapted to contain a cardiothoracic liquid drained from a chest of a patient;
a main unit adapted to contain the components of said drainage device and to measure the cardiothoracic liquid contained in said collection unit, said collection unit being associable with said main unit; and
a connection tube which is configured to be associated with said collection unit, with said main unit and with a drainage tube partially inserted in a thoracic cavity of said patient from which said cardiothoracic liquid is to be drained;
wherein said main unit comprises, inside:
a suction pump, configured to be operatively associated with said connection tube by a pneumatic circuit in order to create a drainage suction pressure and keep said drainage suction pressure adjusted;
a normally closed electric valve, which is inserted in said pneumatic circuit and adapted to ensure a seal of said pneumatic circuit when said suction pump is not moving;
interface means adapted to receive inputs by the user and to supply outputs for a user;
electronic control and management means adapted to control functions of said drainage device; and
pressure sensor means operatively connected to said electronic control and management means for monitoring said drainage suction pressure and intrapleural pressure of said patient.

13. The drainage device according to claim 12, wherein said main unit comprises at least one ultrasonic sensor arranged in a portion of said main unit that is designed to engage a bottom of said collection unit in order to measure a quantity of cardiothoracic liquid collected in said collection unit.

14. The drainage device according to claim 13, wherein said main unit comprises an external enclosure which is hermetic so as to avoid penetration of liquids and dust from outside.

15. The drainage device according to claim 14, wherein said external enclosure is made of impact-resistant plastic material.

16. The drainage device according to claim 14, wherein said external enclosure forms on a back thereof a receptacle for accommodating said collection unit which is provided with interlocking profiles adapted to engage corresponding appendages formed on the bottom of said collection unit so as to form a stable and integral coupling between said main unit and said collection unit; said at least one ultrasonic sensor being arranged in said accommodation receptacle.

17. The drainage device according to claim 16, wherein said main unit comprises two ribs which are arranged laterally to said at least one ultrasonic sensor and protrude in a direction of said collection unit with respect to a resting plane defined by said accommodation receptacle so as to avoid damaging said at least one ultrasonic sensor.

18. The drainage device according to claim 12, wherein said connection tube comprises:
a dual lumen tube which has a main lumen designed to convey the cardiothoracic liquids and a secondary lumen which can have a diameter that is equal to or smaller than that of said main lumen and is dedicated exclusively to evacuation of air that arrives from the chest of the patient;
a connector associated with a first end of said dual lumen tube and provided with a first tapered connector for connection to a drainage tube and with a chamber in which a hydrophobic filtering membrane is present so as to allow the separation of the air from the liquid;
a second connector, associated with a second end of said dual lumen tube which is opposite said first end, and of the quick release type so as to allow a connection of said connection tube to said collection unit so as to allow quick and safe replacement of the collection unit without acting proximate to the drainage tube.

19. The drainage device according to claim 12, wherein said interface means comprises a control keyboard and a display.

20. The drainage device according to claim 12, wherein said suction pump is of the diaphragm type.

21. The drainage device according to claim 12, wherein said main unit comprises electric power storage means for operating of the drainage device while disconnected from an electrical grid.

22. The drainage device according to claim 12, further comprising a wireless telecommunication module configured for a transmission of measured data to an external device.
